(19) United States
(12) Patent Application Publication
(10) Pub. No.: US 2015/0310164 A1
(43) Pub. Date: Oct. 29, 2015

(54) SYSTEM AND METHOD FOR PROCESSING GENOTYPE INFORMATION RELATING TO PAIN PERCEPTION

(71) Applicant: Proove Biosciences, Inc., Annapolis Junction, MD (US)

(72) Inventor: Brian MESHKIN, Fulton, MD (US)

(21) Appl. No.: 14/695,695

(22) Filed: Apr. 24, 2015

Related U.S. Application Data
(60) Provisional application No. 61/984,280, filed on Apr. 25, 2014.

(51) Int. Cl. G06F 19/18; C12Q 1/68

(52) U.S. Cl. CPC G06F 19/18; C12Q 1/6883; C12Q 2600/156

(57) ABSTRACT

There are systems and methods for preparing or using prognostic information about pain perception or performing an assay based on such information. The information may include whether subject has a subject genotype that includes a COMT haplotype diploid, at least two SNP diploids, one or more demographic phenotypes or a combination thereof. The COMT haplotype diploid is a combination of two COMT haplotypes selected from an LPS haplotype, an APS haplotype, a HPS haplotype or a combination thereof in the COMT gene. The at least two SNP diploids are each a combination of two SNP alleles associated with one SNP location in the DRD1 gene, the COMT gene, the OPRK1 gene, the DRD2 gene, the MTHFR gene, the SLC6A4 gene, the HTR2A gene, the DBH gene, the GABRG2 gene, the OPRM1 gene or the SLC6A3 gene.

### PRIORITY

This application claims priority to U.S. Provisional Application No. 61/984,280 entitled “System and Method for Processing Genotype Information Relating to Pain Perception” by Brian Meshkin filed on Apr. 25, 2014, which is incorporated herein by reference in its entirety.

### BACKGROUND

In nature, organisms of the same species usually differ from each other in various aspects such as in their appearance or in one or more aspects of their biology. The differences are often based on genetic distinctions, some of which are called polymorphisms. Polymorphisms are often observed at the level of the whole individual (i.e., phenotype polymorphism), in variant forms of proteins and blood group substances (i.e., biochemical polymorphism), morphological features of chromosomes (i.e., chromosomal polymorphism) or, at the level of DNA, in differences of nucleotides and/or nucleotide sequences (i.e., genetic polymorphism).

Examples of genetic polymorphisms include alleles and haplotypes. An allele is an alternative form of a gene, such as one member of a pair, that is located at a specific position on a chromosome. A haplotype is a combination of alleles, or a combination of single nucleotide polymorphisms (SNPs) on the same chromosome. An example of a genetic polymorphism is an occurrence of one or more genetically alternative phenotypes in a subject due to the presence or absence of an allele or haplotype.

Genetic polymorphisms can play a role in determining differences in an individual's response to a species of drug, a drug dosage or a therapy including one drug or a combination of drugs. Pharmacogenetics and pharmacogenomics are multidisciplinary research efforts to study the relationships among genotypes, gene expression profiles, and phenotypes, as often expressed through the variability between individuals in response to drugs taken. Since the initial sequencing of the human genome, more than a million SNPs have been identified. Some of these SNPs have been used to predict clinical predispositions or responses based upon data gathered from pharmacogenomic studies.

It is well recognized that different patients often exhibit a clinical range of predispositions to perceiving pain. Although many non-genetic factors, such as age, temperament, environment and concomitant therapies commonly influence a patient's perception of pain, it is estimated that genetic differences often account for a substantial amount of variability in different patients' predisposition to perceiving pain.

In pharmacogenomics, there is a desire to identify new polymorphisms and haplotypes to increase sensitivity in the genetic testing of a patient's perception of pain. Of special interest is increased sensitivity in testing for determining genotype-based drug or regimen selections and dose adjustments for pain medications. The genotype information of a patient may help a prescriber understand whether the patient is, or is not, disposed to be receptive to a pain medication.

A patient's genotype information is often utilized to help a prescriber decide between medications based on information associated with a patient's genetic profile (i.e., genotype information). There is a desire to utilize a patient's genotype information in determining the patient's predisposition to perceiving pain. There is also a desire for methods for predicting and/or diagnosing individuals exhibiting irregular predispositions to perceiving pain. Furthermore, there is also a desire to determine genetic information, such as polymorphisms, which may be utilized for predicting variations in pain perception predisposition (PPP) among individuals. There is also a desire to implement systems processing and distributing the detected genetic information in a systematic way. Such genetic information is often useful in providing prognostic information about treatment options for a patient.

Given the foregoing, and to address the above-described limitations, systems and methods are desired for identifying, estimating and/or determining a potential for success of an individual patient's clinical outcome in response to a pain medication based on the patient's genotype information.

### SUMMARY

This summary is provided to introduce a selection of concepts that are further described in the Detailed Description below. The genes, polymorphisms, sequences and sequence identifiers (i.e., SEQ IDs or SEQ ID Numbers) listed or referenced herein are also described in greater detail below in the Detailed Description. This summary is not intended to identify key or essential features of the claimed subject matter. Also, this summary is not intended as an aid in determining the scope of the claimed subject matter.

The present invention meets the above-identified needs by providing systems, methods and computer readable mediums (CRMs) for preparing and utilizing prognostic information associated with a predisposition to a level of pain perception in a patient. The prognostic information is derived from genotype information about a patient's gene profile. The genotype information may be obtained by, inter alia, assaying a sample of genetic material associated with a patient.

The systems, methods and CRMs, according to the principles of the invention, can be utilized to determine prognostic information associated with a risk to a patient based on the patient's pain perception predisposition (PPP). The prognostic information may be used for addressing prescription needs directed to caring for an individual patient. It may also be utilized in managing large healthcare entities, such as insurance providers, utilizing comprehensive business intelligence systems. These and other objects are accomplished by systems, methods and CRMs directed to preparing and utilizing prognostic information associated with pain perception predisposition in a patient, in accordance with the principles of the invention.

According to a first principle of the invention, there is a method for preparing prognostic information about pain perception. The method may include providing information, including DNA information, associated with a human subject. The method may also include determining from the information that the human subject is characterized by at least two demographic phenotypes, the subject has a subject genotype that includes a COMT haplotype diploid, the subject genotype includes at least two SNP diploids or a combination thereof. The method may include detecting, utilizing a detection technology and the information, a presence or absence from the information of the at least two demographic phenotypes, the COMT haplotype diploid and/or the at least two SNP diploids in the subject genotype. The method may include wherein the COMT haplotype diploid is a combination of two COMT haplotypes selected from an LPS haplotype, an APS haplotype, a HPS haplotype or a combination thereof in the COMT gene. The method may include wherein each of the at least two SNP diploids is a combination of two SNP alleles associated with one SNP location. The SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, HTR2A-ANC, HTR2A-HET, and HTR2A-NONA in the HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene, wherein the at least two demographic phenotypes are selected from race, age, gender, depression and/or other mental health demographic phenotypes.

According to a second principle of the invention, there is a system for preparing prognostic information about pain perception. The system may include an interface configured to receive information, including DNA information, associated with a human subject. The system may include a processor configured to determine from the information that the human subject is characterized by at least two demographic phenotypes, the subject has a subject genotype that includes a COMT haplotype diploid, the subject genotype includes at least two SNP diploids or a combination thereof. The processor may be configured to detect, utilizing a detection technology and the information, a presence or absence from the information of the at least two demographic phenotypes, the COMT haplotype diploid and/or the at least two SNP diploids in the subject genotype. The processor may be configured to detect wherein the COMT haplotype diploid is a combination of two COMT haplotypes selected from an LPS haplotype, an APS haplotype, a HPS haplotype or a combination thereof in the COMT gene. The processor may be configured to detect wherein each of the at least two SNP diploids is a combination of two SNP alleles associated with one SNP location. processor may be configured to detect wherein the SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, HTR2A-ANC, HTR2A-HET, and HTR2A-NONA in the HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene. The processor may be configured to detect wherein the at least two demographic phenotypes are selected from race, age, gender, depression and/or other mental health demographic phenotypes.

According to a third principle of the invention, there is a non-transitory computer-readable medium storing computer readable instructions that when executed by a processor perform a method for preparing prognostic information about pain perception. The method may include providing information, including DNA information, associated with a human subject. The method may also include determining from the information that the human subject is characterized by at least two demographic phenotypes, the subject has a subject genotype that includes a COMT haplotype diploid, the subject genotype includes at least two SNP diploids or a combination thereof. The method may include detecting, utilizing a detection technology and the information, a presence or absence from the information of the at least two demographic phenotypes, the COMT haplotype diploid and/or the at least two SNP diploids in the subject genotype. The method may include wherein the COMT haplotype diploid is a combination of two COMT haplotypes selected from an LPS haplotype, an APS haplotype, a HPS haplotype or a combination thereof in the COMT gene. The method may include wherein each of the at least two SNP diploids is a combination of two SNP alleles associated with one SNP location. The SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, HTR2A-ANC, HTR2A-HET, and HTR2A-NONA in the HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene, wherein the at least two demographic phenotypes are selected from race, age, gender, depression and/or other mental health demographic phenotypes.

According to a fourth principle of the invention, there is a method for utilizing prognostic information about pain perception. The method may include receiving information, including DNA information, associated with a human subject, wherein the received information indicates that the human subject is characterized by at least two demographic phenotypes, the subject has a subject genotype that includes a COMT haplotype diploid, the subject genotype includes at least two SNP diploids, or a combination thereof. The method may include wherein the COMT haplotype diploid is a combination of two COMT haplotypes selected from an LPS haplotype, an APS haplotype, a HPS haplotype or a combination thereof in the COMT gene. The method may include wherein each of the at least two SNP diploids is a combination of two SNP alleles associated with one SNP location. The SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, HTR2A-ANC, HTR2A-HET, and HTR2A-NONA in the HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene, wherein the at least two demographic phenotypes are selected from race, age, gender, depression and/or other mental health demographic phenotypes. The method may include processing the received information utilizing a processor. The method may include determining a therapy for the human subject based, at least in part, on the processed information.

According to a fifth principle of the invention, there is a system for utilizing prognostic information about pain perception. The system may include an interface configured to receive information, including DNA information, associated with a human subject, wherein the received information indicates that the human subject is characterized by at least two demographic phenotypes, the subject has a subject genotype that includes a COMT haplotype diploid, the subject genotype includes at least two SNP diploids, or a combination thereof. The system may include a processor configured to process the information including wherein the COMT haplotype diploid is a combination of two COMT haplotypes selected from an LPS haplotype, an APS haplotype, a HPS haplotype or a combination thereof in the COMT gene. The processor may configured to process wherein each of the at least two SNP diploids is a combination of two SNP alleles associated with one SNP location. The SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, HTR2A-ANC, HTR2A-HET, and HTR2A-NONA in the HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene. The processor may configured to process wherein the at least two demographic phenotypes are selected from race, age, gender, depression and/or other mental health demographic phenotypes. The processor may configured to determine a therapy for the human subject based, at least in part, on the processed information.

According to a sixth principle of the invention, there is a non-transitory computer-readable medium storing computer readable instructions that when executed by a processor perform a method for utilizing prognostic information about pain perception. The method may include recieving information, including DNA information, associated with a human subject, wherein the received information indicates that the human subject is characterized by at least two demographic phenotypes, the subject has a subject genotype that includes a COMT haplotype diploid, the subject genotype includes at least two SNP diploids, or a combination thereof. The method may include wherein the COMT haplotype diploid is a combination of two COMT haplotypes selected from an LPS haplotype, an APS haplotype, a HPS haplotype or a combination thereof in the COMT gene. The method may include wherein each of the at least two SNP diploids is a combination of two SNP alleles associated with one SNP location. The SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, HTR2A-ANC, HTR2A-HET, and HTR2A-NONA in the HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene, wherein the at least two demographic phenotypes are selected from race, age, gender, depression and/or other mental health demographic phenotypes. The method may include processing the received information utilizing a processor. The method may include determining a therapy for the human subject based, at least in part, on the processed information.

According to a seventh principle of the invention, there is a method for performing an assay. The method may include providing a sample of genetic material of a human subject. The method may also include determining DNA information from the sample, the DNA information including whether a subject genotype of the subject includes a COMT haplotype diploid, the subject genotype of the subject includes at least two SNP diploids, or a combination thereof, by detecting, utilizing a detection technology and the sample, a presence or absence of the COMT haplotype diploid and/or the at least two SNP diploids in the subject genotype. The method may include wherein the COMT haplotype diploid is a combination of two COMT haplotypes selected from LPS haplotype, APS haplotype, HPS haplotype or a combination thereof in the COMT gene. The method may include wherein each of the at least two SNP diploids is a combination of two SNP alleles associated with one SNP location. The the SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, 5-HTR2A-ANC, 5-HTR2A-HET, and 5-HTR2A-NONA in the 5-HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene.

According to an eighth principle of the invention, there is a system for performing an assay. The system may include an interface configured to receive a sample of genetic material of a human subject. The system may include a processor configured to determine DNA information from the sample, the DNA information including whether a subject genotype of the subject includes a COMT haplotype diploid, the subject genotype of the subject includes at least two SNP diploids, or a combination thereof. The system may be configured to detect, utilizing a detection technology and the sample, a presence or absence of the COMT haplotype diploid and/or the at least two SNP diploids in the subject genotype. The system may be configured to detect wherein the COMT haplotype diploid is a combination of two COMT haplotypes selected from LPS haplotype, APS haplotype, HPS haplotype or a combination thereof in the COMT gene. The system may be configured to detect wherein each of the at least two SNP diploids is a combination of two SNP alleles associated with one SNP location. The system may be configured to detect wherein the SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, 5-HTR2A-ANC, 5-HTR2A-HET, and 5-HTR2A-NONA in the 5-HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene.

According to a ninth principle of the invention, there is a non-transitory computer-readable medium storing computer readable instructions that when executed by a processor perform a method for performing an assay. The method may include providing a sample of genetic material of a human subject. The method may also include determining DNA information from the sample, the DNA information including whether a subject genotype of the subject includes a COMT haplotype diploid, the subject genotype of the subject includes at least two SNP diploids, or a combination thereof, by detecting, utilizing a detection technology and the sample, a presence or absence of the COMT haplotype diploid and/or the at least two SNP diploids in the subject genotype. The method may include wherein the COMT haplotype diploid is a combination of two COMT haplotypes selected from LPS haplotype, APS haplotype, HPS haplotype or a combination thereof in the COMT gene. The method may include wherein each of the at least two SNP diploids is a combination of two SNP alleles associated with one SNP location. The the SNP alleles may be selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, 5-HTR2A-ANC, 5-HTR2A-HET, and 5-HTR2A-NONA in the 5-HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene.

The above summary is not intended to describe each embodiment or every implementation of the present invention. Further features, their nature and various advantages are made more apparent from the accompanying drawings and the following examples and embodiments.

### BRIEF DESCRIPTION OF THE DRAWINGS

Features and advantages of the present invention become more apparent from the detailed description, set forth below, when taken in conjunction with the drawings. In the drawings, like reference numbers indicate identical or functionally similar elements. Additionally, a left-most digit of a reference number identifies a drawing in which the reference number first appears. In addition, it should be understood that the drawings in the figures which highlight an aspect, methodology, functionality and/or advantage of the present invention, are presented for example purposes only. The present invention is sufficiently flexible such that it may be implemented in ways other than shown in the accompanying figures.

FIG. 1 is a block diagram illustrating an assay system which may be utilized for preparing genotype information from a sample of genetic material, according to an example;

FIG. 2 is a block diagram illustrating a prognostic information system which may be utilized for preparing and/or utilizing prognostic information utilizing the genotype information prepared using the assay system of FIG. 1, according to an example;

FIG. 3 is a flow diagram illustrating a prognostic information process for identifying a risk to a patient utilizing the assay system of FIG. 1 and the prognostic information system of FIG. 2, according to an example; and

FIG. 4 is a block diagram illustrating a computer system providing a platform for the assay system of FIG. 1 or the prognostic information system of FIG. 2, according to various examples.

### DETAILED DESCRIPTION

The present invention is useful for preparing and/or utilizing prognostic information about a patient. The prognostic information may be utilized to determine an appropriate therapy for the patient based on their genotype information and their genetic predisposition to perceiving pain. The genetic predisposition may be associated with the selection of a pain medication, a dosage of the pain medication and the utilization of the pain medication in a regimen for treating the patient's medical condition.

The prognostic information may also be utilized for determining dose adjustments that may help a prescriber understand why a patient is or is not responding to a medication dosage, such as an “average” dose. The prognostic information may also be utilized by a prescriber to decide between medications based on the patient's genetic predisposition to perceiving pain. The prognostic information may also be utilized for predicting and/or diagnosing individuals exhibiting a regular or irregular predisposition to perceiving pain. Such genetic information can be very useful in providing prognostic information about treatment options for a patient. The patient may be associated with a medical condition. The patient may also have already been prescribed a medication for treating a medical condition. The present invention has been found to be particularly advantageous for determining a treatment for a patient who may have a regular or irregular predisposition to perceiving pain. While the present invention is not necessarily limited to such applications, various aspects of the invention may be appreciated through a discussion of the various examples in this context, as illustrated through the examples below.

For simplicity and illustrative purposes, the present invention is described by referring mainly to embodiments, principles and examples thereof. In the following description, numerous specific details are set forth in order to provide a thorough understanding of the examples. It is readily apparent however, that the embodiments may be practiced without limitation to these specific details. In other instances, some embodiments have not been described in detail so as not to unnecessarily obscure the description. Furthermore, different embodiments are described below. The embodiments may be used or performed together in different combinations.

The operation and effects of certain embodiments can be more fully appreciated from the examples described below. The embodiments on which these examples are based are representative only. The selection of embodiments is to illustrate the principles of the invention and does not indicate that variables, functions, conditions, techniques, configurations and designs, etc., which are not described in the examples are not suitable for use, or that subject matter not described in the examples is excluded from the scope of the appended claims and their equivalents. The significance of the examples can be better understood by comparing the results obtained therefrom with potential results which can be obtained from tests or trials that may be or may have been designed to serve as controlled experiments and provide a basis for comparison.

Before the systems and methods are described, it is understood that the invention is not limited to the particular methodologies, protocols, systems, platforms, assays, and the like which are described, as these may vary. It is also to be understood that the terminology used herein is intended to describe particular embodiments of the present invention, and is in no way intended to limit the scope of the present invention as set forth in the appended claims and their equivalents.

Throughout this disclosure, various publications, such as patents and published patent specifications, are referenced by an identifying citation. The disclosures of these publications are hereby incorporated by reference in their entirety into the present disclosure in order to more fully describe the state of the art to which the invention pertains.

The practice of the present invention employs, unless otherwise indicated, conventional techniques of molecular biology, microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al., U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)); MANIPULATING THE MOUSE EMBRYO: A LABORATORY MANUAL 3rd edition (Cold Spring Harbor Laboratory Press, Cold Spring Harbor, N.Y. (2002)).

### DEFINITIONS

As used herein, certain terms have the following defined meanings. As used herein, the singular form “a,” “an” and “the” includes the singular and plural references unless the context clearly dictates otherwise. For example, the term “a cell” includes a single cell and a plurality of cells, including mixtures thereof.

As used herein, the terms “based on”, “comprises”, “comprising”, “includes”, “including”, “has”, “having” or any other variation thereof, are intended to cover a non-exclusive inclusion. For example, a system, process, method, article, or apparatus that comprises a list of elements is not necessarily limited to only those elements but may include other elements not expressly listed or inherent to such system, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

All numerical designations, e.g., pH, temperature, time, concentration, and molecular weight, including ranges, are approximations which may be varied (+) or (−) by minor increments, such as, of 0.1. It is to be understood, although not always explicitly stated, that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known to those of ordinary skill in the art.

The term “allele”. which is used interchangeably herein with the term “allelic variant” refers to alternative forms of a gene or any portions thereof. Alleles may occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions and insertions of nucleotides. An allele of a gene can also be an ancestral form of a gene or a form of a gene containing a mutation.

The term “haplotype” refers to a combination of alleles on a chromosome or a combination of SNPs within an allele on one chromosome. The alleles or SNPs may or may not be at adjacent locations (loci) on a chromosome. A haplotype may be at one locus, at several loci or an entire chromosome.

The term “ancestral,” when applied to describe an allele in a human, refers to an allele of a gene that is the same or nearest to a corresponding allele appearing in the corresponding gene of the chimpanzee genome. Often, but not always, a human ancestral allele is the most prevalent human allelic variant appearing in nature—i.e., the allele with the highest gene frequency in a population of the human species.

The term “wild-type,” when applied to describe an allele, refers to an allele of a gene which, when it is present in two copies in a subject, results in a wild-type phenotype. There can be several different wild-type alleles of a specific gene. Also, nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region may include, for example, a single nucleotide polymorphism (SNP), the identity of which differs in the different alleles by a single nucleotide at a locus in the polymorphic region of the gene. In another example, a polymorphic region may include a deletion or substitution of one or more nucleotides at a locus in the polymorphic region of the gene.

The expression “amplification of polynucleotides” includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, a PCR procedure is a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively, the amplified sequence(s) may be cloned prior to sequence analysis. Methods for direct cloning and sequence analysis of enzymatically amplified genomic segments are known in the art.

The term “encode”, as it is applied to polynucleotides, refers to a polynucleotide which is said to “encode” a polypeptide. The polynucleotide is transcribed to produce mRNA, which is then translated into the polypeptide and/or a fragment thereof by cell machinery. An antisense strand is the complement of such a polynucleotide, and the encoding sequence can be deduced therefrom.

As used herein, the term “gene” or “recombinant gene” refers to a nucleic acid molecule comprising an open reading frame and including at least one exon and optionally an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A “related” or “homologous” sequence shares identity with a comparative sequence, such as 100%, at least 99%, at least 95%, at least 90%, at least 80%, at least 70%, at least 60%, at least 50%, at least 40%, at least 30%, at least 20%, or at least 10%. An “unrelated” or “non-homologous” sequence shares less identity with a comparative sequence, such as less than 95%, less than 90%, less than 80%, less than 70%, less than 60%, less than 50%, less than 40%, less than 30%, less than 20%, or less than 10%.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “isolated” as used herein with respect to nucleic acids, such as DNA or RNA, refers to molecules separated from other DNAs or RNAs, respectively, which are present in a natural source of a macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the term “nucleic acid” refers to polynucleotides such as deoxyribonucleic acid (DNA), and, where appropriate, ribonucleic acid (RNA). The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides.

Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine”, “cytidine”, “guanosine”, and “thymidine” are used. It is understood that if the nucleic acid is RNA, it includes nucleotide(s) having a uracil base that is uridine.

The terms “oligonucleotide” or “polynucleotide”, or “portion,” or “segment” thereof refer to a stretch of polynucleotide residues which may be long enough to use in PCR or various hybridization procedures to identify or amplify identical or related parts of mRNA or DNA molecules. The polynucleotide compositions described herein may include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications can include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). This may also include synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The phrase “genetic profile” is used interchangeably with “genotype information” and refers to part or all of an identified genotype of a subject and may include one or more polymorphisms in one or more genes of interest. A genetic profile may not be limited to specific genes and polymorphisms described herein, and can include any number of other polymorphisms, gene expression levels, polypeptide sequences, or other genetic markers that are associated with a subject or patient.

The term “patient” refers to an individual waiting for or under medical care and treatment, such as a treatment for medical condition. While the disclosed methods are designed for human patients, such methods are applicable to any suitable individual, which includes, but is not limited to, a mammal, such as a mouse, rat, rabbit, hamster, guinea pig, cat, dog, goat, cow, horse, pig, and simian. Human patients include male and female patients of any ethnicity. The term “treating” as used herein is intended to encompass curing as well as ameliorating at least one symptom of a condition or disease.

The nucleic acid codes utilized herein include: A for Adenine, C for Cytosine, G for Guanine, T for Thymine, U for Uracil, R for A or G, Y for C, T or U, K for G, T or U, and M for A or C.

As used herein, the terms “drug”, “medication”, and “therapeutic compound” or “compound” are used interchangeably and refer to any chemical entity, pharmaceutical, drug, biological, and the like that can be used to treat or prevent a disease, illness, condition, or disorder of bodily function. A drug may comprise both known and potentially therapeutic compounds. A drug may be determined to be therapeutic by screening using the screening known to those having ordinary skill in the art. A “known therapeutic compound” or “medication” refers to a therapeutic compound that has been shown (e.g., through animal trials or prior experience with administration to humans) to be effective in such treatment. Examples of drugs include, but are not limited to peptides, polypeptides, synthetic organic molecules, naturally occurring organic molecules, nucleic acid molecules, and combinations thereof.

### EXEMPLARY EMBODIMENTS

The biological basis for an outcome in a specific patient following a treatment with pain medication is subject to, inter alia, the patient's genetic predisposition to perceiving pain. It has been determined that select polymorphisms, including single nucleotide permutations, haplotypes and sex-based phenotypes. The polymorphisms may be utilized to generate genotype information. The genotype information may be utilized to generate prognostic information. The prognostic information may be utilized in determining treatment options for the patient. The prognostic information is based on the patient's genetic predisposition to perceiving pain. The prognostic information may also be utilized in determining an expected outcome of a treatment of an individual, such as a treatment with pain a medication.

When a genetic marker such as a polymorphism is used as a basis for determining a treatment for a patient, as described herein, the genetic marker may be measured before or during treatment. The prognostic information obtained may be used by a clinician in assessing any of the following: (a) a probable or likely suitability of an individual to initially receive treatment(s); (b) a probable or likely unsuitability of an individual to initially receive treatment(s); (c) a responsiveness to treatment; (d) a probable or likely suitability of an individual to continue to receive treatment(s); (e) a probable or likely unsuitability of an individual to continue to receive treatment(s); (f) adjusting dosage; (g) predicting likelihood of clinical benefits. As understood by one of skill in the art, measurement of a genetic marker or polymorphism in a clinical setting can be an indication that this parameter may be used as a basis for initiating, continuing, adjusting and/or ceasing administration of treatment, such as described herein.

Select polymorphisms have been identified which may be utilized for providing prognostic information, according to the principles of the invention. These findings were correlated with various magnitudes of a positive or negative predispositions to pain perception. Accordingly, assaying the genotype at these markers may be utilized to generate prognostic information which may be utilized to predict the expected outcome of treating the patient with a pain medication based on the expected predisposition of the patient to perceiving pain. Clinicians prescribing pain medication and other medications may utilize the prognostic information to improve therapeutic decisions and to avoid treatment failures.

Many of the known human single nucleotide permutations (SNPs) are catalogued by the National Center for Biotechnology Information (NCBI) in the Reference SNP (i.e., “refSNP”) database maintained by NCBI. The Reference SNP database is a polymorphism database (dbSNP) which includes single nucleotide polymorphisms and related polymorphisms, such as deletions and insertions of one or more nucleotides. The database is a public-domain archive maintained by NCBI for a broad collection of simple genetic polymorphisms and can be accessed at http://www.ncbi.nlm.nih.gov/snp.

DNA polymorphisms have been identified which may be utilized according to the principles of the invention include SNPs and haplotypes associated with genetic markers in several genes. The genes include the respective genes encoding the Dopamine D1 Receptor (abbreviated DRD1), the Catechol-O-Methyltransferase enzyme (abbreviated COMT), the Serotonin Transporter gene (also known as Solute Carrier Family 6 (Neurotransmitter Transporter), member 4 and abbreviated SLC6A4), the Human Kappa Opioid Receptor (abbreviated OPRK1), the Dopamine D2 Receptor (abbreviated DRD2), the Methylenetetrahydrofolate Reductase enzyme (abbreviated MTHFR), the 5-Hydroxytryptamine (Serotonin) Receptor 2A, G Protein-Coupled (abbreviated HTR2A), the Dopamine Beta-Hydroxylase enzyme (abbreviated DBH), the (GABA)-A Receptor, gamma 2 subunit (abbreviated GABRG2), the Opioid Receptor, Mu 1 (abbreviated OPRM1), and the Dopamine Transporter (also known as Solute Carrier Family 6 (Neurotransmitter Transporter), member 3, abbreviated SLC6A3 or DAT).

The DNA polymorphisms which have been identified as active for predicting a genetic pain perception predisposition (PPP), are SNP Diploid Polymorphisms. In the identified SNP diploid polymorphisms, the pain perception impact may vary depending upon the active allele of a SNP in a chromosome of a gene as well as the zygosity of the SNP diploid at the locus of the SNP on the chromosome. The SNP diploid polymorphisms identified as predictive of pain perception predisposition are listed in Table 1 below.

TABLE 1*
Identification of SNP Diploid Polymorphisms

| | | SNP Diploid | | DNA Context | |
|---|---|---|---|---|---|
| No. | rs# | ID** | Zygosity | Sequence for Active SNP(s)*** | SEQ ID |
| 1 | rs4532 | DRD1-ANC | homozygous | AGGGGCTCTGACACCCCTCAAGTTCC[T]AA | SEQ ID No: 1 |
| | | | | GCAGGGAATAGGGGTCAGTCAGA | |
| 2 | rs4532 | DRD1-HET | heterozygous | AGGGGCTCTGACACCCCTCAAGTTCC[C/T] | SEQ ID No: 2 |
| | | | | AAGCAGGGAATAGGGGTCAGTCAGA | |
| 3 | rs4532 | DRD1-NONA | homozygous | AGGGGCTCTGACACCCCTCAAGTTCC[C]AA | SEQ ID No: 3 |
| | | | | GCAGGGAATAGGGGTCAGTCAGA | |
| 4 | rs4633 | COMT(1)-ANC | homozygous | CCAAGGAGCAGCGCATCCTGAACCA[C]GTG | SEQ ID No: 4 |
| | | | | CTGCAGCATGCGGAGCCCGGGA | |
| 5 | rs4633 | COMT(1)-HET | heterozygous | CCAAGGAGCAGCGCATCCTGAACCA[C/T]G | SEQ ID No: 5 |
| | | | | TGCTGCAGCATGCGGAGCCCGGGA | |
| 6 | rs4633 | COMT(1)-NONA | homozygous | CCAAGGAGCAGCGCATCCTGAACCA[T]GTG | SEQ ID No: 6 |
| | | | | CTGCAGCATGCGGAGCCCGGGA | |
| 7 | rs4680 | COMT(2)-ANC | homozygous | CCCAGCGGATGGTGGATTTCGCTGGC[G]TG | SEQ ID No: 7 |
| | | | | AAGGACAAGGTGTGCATGCCTGA | |
| 8 | rs4680 | COMT(2)-HET | heterozygous | CCCAGCGGATGGTGGATTTCGCTGGC[G/A] | SEQ ID No: 8 |
| | | | | TGAAGGACAAGGTGTGCATGCCTGA | |
| 9 | rs4680 | COMT(2)-NONA | homozygous | CCCAGCGGATGGTGGATTTCGCTGGC[A]T | SEQ ID No: 9 |
| | | | | GAAGGACAAGGTGTGCATGCCTGA | |
| 10 | rs4818 | COMT(3)-ANC | homozygous | GCCTGCTGTCACCAGGGGCGAGGCT[G]AT | SEQ ID No: 10 |
| | | | | CACCATCGAGATCAACCCCGACT | |
| 11 | rs4818 | COMT(3)-HET | heterozygous | GCCTGCTGTCACCAGGGGCGAGGCT[G/C/ | SEQ ID No: 11 |
| | | | | T]ATCACCATCGAGATCAACCCCGACT | |
| 12 | rs4818 | COMT(3)-NONA | homozygous | GCCTGCTGTCACCAGGGGCGAGGCT[C/T] | SEQ ID No: 12 |
| | | | | ATCACCATCGAGATCAACCCCGACT | |
| 13 | rs6269 | COMT(4)-ANC | homozygous | GCATTTCTGAACCTTGCCCCTCTGC[G]AA | SEQ ID No: 13 |
| | | | | CACAAGGGGGCGATGGTGGCACT | |
| 14 | rs6269 | COMT(4)-HET | heterozygous | GCATTTCTGAACCTTGCCCCTCTGC[G/A] | SEQ ID No: 14 |
| | | | | AACACAAGGGGGCGATGGTGGCACT | |
| 15 | rs6269 | COMT(4)-NONA | homozygous | GCATTTCTGAACCTTGCCCCTCTGC[A]A | SEQ ID No: 15 |
| | | | | ACACAAGGGGGCGATGGTGGCACT | |
| 16 | rs25531 | SLC6A4*-ANC | homozygous | CCTCGCGGCATCCCCCCTGCACCCCC[A] | SEQ ID No: 16 |
| | | | | GCATCCCCCCTGCAGCCCCCCCAGC | |
| 17 | rs25531 | SLC6A4*-HET | heterozygous | CCTCGCGGCATCCCCCCTGCACCCCC[A/ | SEQ ID No: 17 |
| | | | | G]GCATCCCCCCTGCAGCCCCCCCAGC | |
| 18 | rs25531 | SLC6A4*-NONA | homozygous | CCTCGCGGCATCCCCCCTGCACCCCC[G] | SEQ ID No: 18 |
| | | | | GCATCCCCCCTGCAGCCCCCCCAGC | |
| 19 | rs1051660 | OPRK1-ANC | homozygous | CCGATCCAGATCTTCCGCGGGGAGCC[G] | SEQ ID No: 19 |
| | | | | GGCCCTACCTGCGCCCCGAGCGCCT | |
| 20 | rs1051660 | OPRK1-HET | heterozygous | CCGATCCAGATCTTCCGCGGGGAGCC[G/ | SEQ ID No: 20 |
| | | | | A/T]GGCCCTACCTGCGCCCCGAGCGCCT | |
| 21 | rs1051660 | OPRK1 -NONA | homozygous | CCGATCCAGATCTTCCGCGGGGAGCC[A/ | SEQ ID No: 21 |
| | | | | T]GGCCCTACCTGCGCCCCGAGCGCCT | |
| 22 | rs1800497 | DRD2-ANC | homozygous | CTGGACGTCCAGCTGGGCGCCTGCCT[T] | SEQ ID No: 22 |
| | | | | GACCAGCACTTTGAGGATGGCTGTG | |
| 23 | rs1800497 | DRD2-HET | heterozygous | CTGGACGTCCAGCTGGGCGCCTGCCT[C/ | SEQ ID No: 23 |
| | | | | T]GACCAGCACTTTGAGGATGGCTGTG | |
| 24 | rs1800497 | DRD2-NONA | homozygous | CTGGACGTCCAGCTGGGCGCCTGCCT[C] | SEQ ID No: 24 |
| | | | | GACCAGCACTTTGAGGATGGCTGTG | |
| 25 | rs1801133 | MTHFR-ANC | homozygous | CTTGAAGGAGAAGGTGTCTGCGGGAG[C] | SEQ ID No: 25 |
| | | | | CGATTTCATCATCACGCAGCTTTTC | |
| 26 | rs1801133 | MTHFR-HET | heterozygous | CTTGAAGGAGAAGGTGTCTGCGGGAG[C/ | SEQ ID No: 26 |
| | | | | T]CGATTTCATCATCACGCAGCTTTTC | |
| 27 | rs1801133 | MTHER-NONA | homozygous | CTTGAAGGAGAAGGTGTCTGCGGGAG[T] | SEQ ID No: 27 |
| | | | | CGATTTCATCATCACGCAGCTTTTC | |
| 28 | rs140701 | SLC6A4-ANC | homozygous | CACATAAGGTCTTGTGATGAGAATT[G]T | SEQ ID No: 28 |
| | | | | AACTGTTGTTGTGGCTGAGTTTTC | |
| 29 | rs140701 | SLC6A4-HET | heterozygous | CACATAAGGTCTTGTGATGAGAATT[A/G] | SEQ ID No: 29 |
| | | | | TAACTGTTGTTGTGGCTGAGTTTTC | |
| 30 | rs140701 | SLC6A4-NONA | homozygous | CACATAAGGTCTTGTGATGAGAATT[A]TA | SEQ ID No: 30 |
| | | | | ACTGTTGTTGTGGCTGAGTTTTC | |
| 31 | rs7997012 | HTR2A-ANC | homozygous | TGCCATTATCTTCAAAGACTTAATT[G]AC | SEQ ID No: 31 |
| | | | | AATATTTGTCACTTGCCTATGCA | |
| 32 | rs7997012 | HTR2AHET | heterozygous | TGCCATTATCTTCAAAGACTTAATT[A/G] | SEQ ID No: 32 |
| | | | | ACAATATTTGTCACTTGCCTATGCA | |
| 33 | rs7997012 | HTR2A-NONA | homozygous | TGCCATTATCTTCAAAGACTTAATT[A] | SEQ ID No: 33 |
| | | | | ACAATATTTGTCACTTGCCTATGCA | |
| 34 | rs1611115 | DBH-ANC | homozygous | AAGGCAGCTGCCCTCAGTCTACTTG[C]GG | SEQ ID No: 34 |
| | | | | GAGAGGACAGGAGGGAGAGGTGC | |
| 35 | rs1611115 | DBH-HET | heterozygous | AAGGCAGCTGCCCTCAGTCTACTTG[C/T] | SEQ ID No: 35 |
| | | | | GGGAGAGGACAGGAGGGAGAGGTGC | |
| 36 | rs1611115 | DBH-NONA | homozygous | AAGGCAGCTGCCCTCAGTCTACTTG[T]GG | SEQ ID No: 36 |
| | | | | GAGAGGACAGGAGGGAGAGGTGC | |
| 37 | rs211014 | GABRG2-ANC | homozygous | GCAGGCTAAGGCTCAGCAGTTTGGG[C]TC | SEQ ID No: 37 |
| | | | | CAAGATGAAAACAGCATGTATGA | |
| 38 | rs211014 | GABRG2-HET | heterozygous | GCAGGCTAAGGCTCAGCAGTTTGGG[A/C] | SEQ ID No: 38 |
| | | | | TCCAAGATGAAAACAGCATGTATGA | |
| 39 | rs211014 | GABRG2-NONA | homozygous | GCAGGCTAAGGCTCAGCAGTTTGGG[A]TC | SEQ ID No: 39 |
| | | | | CAAGATGAAAACAGCATGTATGA | |
| 40 | rs1799971 | OPRM1-ANC | homozygous | GGTCAACTTGTCCCACTTAGATGGC[A]AC | SEQ ID No: 40 |
| | | | | CTGTCCGACCCATGCGGTCCGAA | |
| 41 | rs1799971 | OPRM1-HET | heterozygous | GGTCAACTTGTCCCACTTAGATGGC[A/G] | SEQ ID No: 41 |
| | | | | ACCTGTCCGACCCATGCGGTCCGAA | |
| 42 | rs1799971 | OPRM1-NONA | homozygous | GGTCAACTTGTCCCACTTAGATGGC[G]AC | SEQ ID No: 42 |
| | | | | CTGTCCGACCCATGCGGTCCGAA | |
| 43 | rs27072 | SLC6A3-ANC | homozygous | AGTGCCCCTGGGGCAGCCTCAGAGC[C]GG | SEQ ID No: 43 |
| | | | | GAGCAGGGAGCAGGGAGGGAGGG | |
| 44 | rs27072 | SLC6A3-HET | heterozygous | AGTGCCCCTGGGGCAGCCTCAGAGC[C/T] | SEQ ID No: 44 |
| | | | | GGGAGCAGGGAGCAGGGAGGGAGGG | |
| 45 | rs27072 | SLC6A3-NONA | homozygous | AGTGCCCCTGGGGCAGCCTCAGAGC[T]GG | SEQ ID No: 45 |
| | | | | GAGCAGGGAGCAGGGAGGGAGGG | |

* Unless otherwise indicated, the context sequences are in FASTA format, as presented by NCBI within the rs cluster report identified by “rs#” in the NCBI SNP reference database accessible at http://www.ncbi.nlm.nih. gov/snp.
** The naming conventions for the SNP Diploid Polymorphisms indicate the diploid is either -ANC (homozygous for the ancestral SNP), -HET (heterozygous as including one ancestral and one non-ancestral SNP in the diploid), or -NONA (homozygous for the non-ancestral SNP).
*** Brackets (i.e., “[...]”) appear within each context sequence to indicate the location (i.e., the “polymorphism marker” or “marker”) of the polymorphic region in the context sequence.

In Table 1, the active polymorphisms are the the diploid pair of alleles associated with “SNP markers” called “rs numbers” in the refSNP database. A SNP marker in dbSNP references a SNP cluster report identification number (i.e., a “rs number”) in the refSNP database. The context sequences shown in Table 1 include the allelic variant(s) and the zygosity of the diploid pair identified as active for providing prognostic information according to the principles of the invention. The context sequences include the active polymorphism SNP located in the relevant region of the gene. The context sequences also include a number of nucleotide bases flanking the active polymorphism SNP in the relevant region of the gene. In the context sequences shown in Table 1, the polymorphic SNP location is shown in brackets within the context sequence for identification purposes. Table 1 also show the rs cluster report number (i.e., the “rs number”) associated with the active polymorphism SNP in dbSNP maintained by NCBI. The other alleles identified as “Inactive SNPs”, according to the principles of the invention, are also associated with the cluster reports for rs numbers in the refSNP database.

Studies have been conducted and it has been determined that SNP diploid polymorphisms identified in Table 1 are predictive of a differential predisposition to perceiving pain by a patient having one or more of SNP diploid polymorphisms compared with an “average” patient not having the SNP diploid polymorphisms. Certain SNP diploid polymorphisms in Table 1 are associated with a patient having an elevated pain perception predisposition (i.e., predisposed to higher perception of pain and/or lower tolerance to perceived pain). Certain SNP diploid polymorphisms in Table 1 are associated with a patient having a reduced pain perception predisposition (i.e., predisposed to lower perception of pain and/or higher tolerance to perceived pain).

In the studies, subjects were genotyped using a RealTime PCR TaqMan assay from Proove Medical Laboratories (Irvine, Calif.). Subjects completed a Pain Visual Analogue Scale (VAS) rating their perception of pain on a scale. Low pain perception was defined as a lower score. Moderate pain perception was defined as a middle score. High pain perception was defined as a high score. A multinomial logistic regression analysis was performed using SPSS. Statistical significance was found among the different variants to objectively stratify pain perception. Exemplary odds ratios (OR) were determined to quantify how strongly the presence or absence of an identified SNP diploid polymorphism was associated with the presence or absence of a reduced pain perception (PP), neutral PP or elevated PP. According to the Exemplary ORs generated in this example, the net effect of the added ORs of an average person is equivalent to zero. The results of the mathematical analysis on the identified SNP diploid polymorphisms are listed in Table 2 below.

TABLE 2
Results for SNP Diploid Polymorphisms

| | | General Impact | Exemplary Description of | Exem- |
|---|---|---|---|---|
| | SNP Diploid | to Pain | Pain Perception | plary |
| No.* | Polymorphism ID | Perception (PP) | Predisposition (PPP) | OR |
| 1 | DRD1-ANC | Reduced PP | Moderate Perception/Moderate to Low Perception (50%) | −0.6445 |
| 2 | DRD1-HET | Nuetral | None | 0 |
| 3 | DRD1-NONA | Elevated PP | High Perception/Low Tolerance | +1.289 |
| 7 | COMT(2)-ANC | Reduced PP | Moderate Perception/Moderate to Low Perception (50%) | −0.8315 |
| 8 | COMT(2)-HET | Nuetral | None | 0 |
| 9 | COMT(2)-NONA | Elevated PP | High Perception/Low Tolerance | +1.663 |
| 16 | SLC6A4*-ANC | Nuetral | None | 0 |
| 17 | SLC6A4*-HET | Reduced PP | Low Perception/High Tolerance | −2.810 |
| 18 | SLC6A4*-NONA | Elevated PP | Moderate Perception/Moderate to High Perception (50%) | +1.405 |
| 19 | OPRK1-ANC | Nuetral | None | 0 |
| 20 | OPRK1-HET | Elevated PP | High Perception/Low Tolerance | +1.312 |
| 21 | OPRK1-NONA | Reduced PP | Moderate Perception/Moderate to Low Perception (50%) | −0.6560 |
| 22 | DRD2-ANC | Elevated PP | High Perception/Low Tolerance | +1.293 |
| 23 | DRD2-HET | Nuetral | None | 0 |
| 24 | DRD2-NONA | Reduced PP | Moderate Perception/Moderate to Low Perception (50%) | −0.6465 |
| 25 | MTHFR-ANC | Neutral | None | 0 |
| 26 | MTHFR-HET | Elevated PP | High Perception/Low Tolerance | −1.633 |
| 27 | MTHFR-NONA | Moderate PP | Moderate Perception/Moderate Tolerance | +1.633 |
| 28 | SLC6A4-ANC | Moderate PP | Moderate Perception/Moderate Tolerance | +1.289 |
| 29 | SLC6A4-HET | Neutral | None | 0 |
| 30 | SLC6A4-NONA | Elevated PP | High Perception/Low Tolerance | −1.289 |
| 31 | 5-HTR2A-ANC | Elevated PP | High Perception/Low Tolerance | −1.290 |
| 32 | 5-HTR2A-HET | Neutral | None | 0 |
| 33 | 5-HTR2A-NONA | Moderate PP | Moderate Perception/Moderate Tolerance | +1.290 |
| 34 | DBH-ANC | Moderate PP | Moderate Perception/Moderate Tolerance | +1.663 |
| 35 | DBH-HET | Neutral | None | 0 |
| 36 | DBH-NONA | Elevated PP | High Perception/Low Tolerance | −1.663 |
| 37 | GABRG2-ANC | Elevated PP | High Perception/Low Tolerance | +1.000 |
| 38 | GABRG2-HET | Neutral | None | 0 |
| 39 | GABRG2-NONA | Reduced PP | Low Perception/High Tolerance | −1.000 |
| 40 | OPRM1-ANC | Elevated PP | High Perception/Low Tolerance | +2.520 |
| 41 | OPRM1-HET | Neutral | None | 0 |
| 42 | OPRM1-NONA | Reduced PP | Low Perception/High Tolerance | −2.520 |
| 43 | SLC6A3-ANC | Neutral | None | 0 |
| 44 | SLC6A3-HET | Reduced PP | Low Perception/High Tolerance | −3.000 |
| 45 | SLC6A3-NONA | Elevated PP/Moderate PP | High-Moderate Perception/Low-Moderate Tolerance | +3.000 |

*Reference Nos. and IDs in Table 2 coincides with same Reference Nos. and IDs appearing in Table 1.

Several demographic phenotypes of the subjects in the studies were also analyzed to determine correlations with higher or lower pain perception. A multinomial logistic regression analysis was performed using SPSS. The demographic phenotypes analyzed were Race, Gender, Age and whether a subject had a medical history or diagnosis of Depression, and/or another mental health phenotype such as Anxiety Disorder, Attention Deficit Disorder (ADD), Obsessive Compulsive Disorder (OCD), Bipolar Disorder and Schizophrenia. Statistical significance was found among the different demographic phenotypes to objectively stratify pain perception according to the different characteristics of a subject. Exemplary odds ratios (OR) were determined to quantify how strongly the presence or absence a sex phenotype was associated with the presence or absence of a reduced pain perception (PP), neutral PP or elevated PP. The results of the mathematical analysis on the impact of specific demographic phenotypes on pain pereception predisposition are listed in Tables 3A-3I below.

TABLE 3A
Results for Race Demographic Phenotype

| | | General Impact | Exemplary Description | Exem- |
|---|---|---|---|---|
| | Race | to Pain | of Pain Perception | plary |
| No. | Polymophism | Perception (PP) | Predisposition (PPP) | OR |
| 1 | Hispanic | Elevated PP | High Perception/Low | +1.000 |
| | | | Tolerance | |
| 2 | Black | Elevated PP | High Perception/Low | +1.000 |
| | | | Tolerance | |
| 3 | Caucasian | Reduced PP | Low Perception/High | −1.000 |
| | | | Tolerance | |

TABLE 3B
Results for Sex Demographic Phenotype (P value + 0.036)

| | | General Impact | Exemplary Description | Exem- |
|---|---|---|---|---|
| | Sex | to Pain | of Pain Perception | plary |
| No. | Polymophism | Perception (PP) | Predisposition (PPP) | OR |
| 1 | Female | Elevated PP | High Perception/Low | +1.345 |
| | | | Tolerance | |
| 2 | Male | Reduced PP | Low Perception/High | −1.345 |
| | | | Tolerance | |

TABLE 3C
Results for Age Demographic Phenotype
(age correlation at age 60)

| | | General Impact | Exemplary Description | Exem- |
|---|---|---|---|---|
| | Age | to Pain | of Pain Perception | plary |
| No. | Polymophism | Perception (PP) | Predisposition (PPP) | OR |
| 1 | 18 to 59 | Elevated PP | High Perception/Low | +1.000 |
| | | | Tolerance | |
| 2 | 60 or older | Reduced PP | Low Perception/High | −1.000 |
| | | | Tolerance | |

TABLE 3D
Results for Depression Demographic Phenotype
(medical history or diagnosis)

| | | General Impact | Exemplary Description | Exem- |
|---|---|---|---|---|
| | Depression | to Pain | of Pain Perception | plary |
| No. | Polymophism | Perception (PP) | Predisposition (PPP) | OR |
| 1 | Identified | Elevated PP | High Perception/Low | +1.000 |
| | | | Tolerance | |
| 2 | Not | Nuetral | None | 0 |

TABLE 3E
Results for Anxiety Disorder Demographic
Phenotype (medical history or diagnosis)

| | | General Impact | Exemplary Description | Exem- |
|---|---|---|---|---|
| | Anxiety | to Pain | of Pain Perception | plary |
| No. | Polymophism | Perception (PP) | Predisposition (PPP) | OR |
| 1 | Identified | Elevated PP | High Perception/Low | +1.000 |
| | | | Tolerance | |
| 2 | Not | Nuetral | None | 0 |

TABLE 3F
Results for ADD Demographic Phenotype
(medical history or diagnosis)

| | | General Impact | Exemplary Description | Exem- |
|---|---|---|---|---|
| | ADD | to Pain | of Pain Perception | plary |
| No. | Polymophism | Perception (PP) | Predisposition (PPP) | OR |
| 1 | Identified | Elevated PP | High Perception/Low | +1.000 |
| | | | Tolerance | |
| 2 | Not | Nuetral | None | 0 |

TABLE 3G
Results for OCD Demographic Phenotype
(medical history or diagnosis)

| | | General Impact | Exemplary Description | Exem- |
|---|---|---|---|---|
| | OCD | to Pain | of Pain Perception | plary |
| No. | Polymophism | Perception (PP) | Predisposition (PPP) | OR |
| 1 | Identified | Elevated PP | High Perception/Low | +1.000 |
| | | | Tolerance | |
| 2 | Not | Nuetral | None | 0 |

TABLE 3H
Results for Bipolar Disorder Demographic
Phenotype (medical history or diagnosis)

| | | General Impact | Exemplary Description | Exem- |
|---|---|---|---|---|
| | BPD | to Pain | of Pain Perception | plary |
| No. | Polymophism | Perception (PP) | Predisposition (PPP) | OR |
| 1 | Identified | Elevated PP | High Perception/Low | +1.000 |
| | | | Tolerance | |
| 2 | Not | Nuetral | None | 0 |

TABLE 3I
Results for Schizophrenia Demographic Phenotype
(medical history or diagnosis)

| | | General Impact | Exemplary Description | Exem- |
|---|---|---|---|---|
| | Schizophrenia | to Pain | of Pain Perception | plary |
| No. | Polymophism | Perception (PP) | Predisposition (PPP) | OR |
| 1 | Identified | Elevated PP | High Perception/Low | +1.000 |
| | | | Tolerance | |
| 2 | Not | Nuetral | None | 0 |

Certain haplotypes of the COMT gene are known in the art to be associated with pain sensitivity. However, the COMT haplotypes have not been correlated in a combined analysis involving other genetic and characteristic phenotypes as described herein. The COMT haplotypes associated with pain sensitivity have been identified as predictive of pain perception predisposition and can be correlated with the other genetic and characteristic phenotypes as described above. The COMT haplotypes are listed in Table 4 below.

TABLE 4
Identification of COMT haplotypes

| | COMT | | | | |
|---|---|---|---|---|---|
| | Haplotype | COMT Haplotype Structure | | | |
| No. | Polymorphism | rs6269-SNP | rs4633-SNP | rs4818-SNP | rs4680-SNP |
| 1 | LPS | . . . [G] . . . | . . . [C] . . . | . . . [G] . . . | . . . [G] . . . |
| 2 | APS | . . . [A] . . . | . . . [T] . . . | . . . [C] . . . | . . . [A] . . . |
| 3 | HPS | . . . [A] . . . | . . . [C] . . . | . . . [C] . . . | . . . [G] . . . |

The COMT haplotypes of the subjects in the studies were analyzed to determine correlations with higher or lower pain perception. A multinomial logistic regression analysis was performed using SPSS. Statistical significance was found for the presence of COMT haplotype to objectively stratify pain perception based on the presence of a COMT haplotype. Exemplary odds ratios (OR) were determined to quantify the impact of a COMT haplotype to be associated with the presence or absence of a reduced pain perception (PP), neutral PP or elevated PP. The results of the mathematical analysis on the impact of COMT haplotype on pain perception predisposition are listed in Table 5 below.

TABLE 5
Results for COMT Haplotype Polymorphisms

| | COMT | General Impact | Exemplary Description | Exem- |
|---|---|---|---|---|
| | Haplotype | to Pain | of Pain Perception | plary |
| No. | Polymorphism | Perception (PP) | Predisposition (PPP) | OR |
| 1 | LPS | Reduced PP | Low Perception/High | −1.000 |
| | | | Tolerance | |
| 2 | APS | Neutral | Moderate Perception/ | 0 |
| | | | Moderate Tolerance | |
| 3 | HPS | Elevated PP | High Perception/Low | +1.000 |
| | | | Tolerance | |

Furthermore, the COMT haplotype information associated with a subject has been further differentiated in Table 5 (i.e., LPS, APS and HPS) based on the diplotype (i.e., the diploid pair) of the COMT haplotypes in a subject (i.e., the pair of COMT haplotypes appearing in the subject's COMT gene). The COMT haplotype diploid polymorphism of the subjects in the studies were analyzed to determine correlations with higher or lower pain perception. A multinomial logistic regression analysis was performed using SPSS. Statistical significance was found for the presence of specific COMT haplotype diploid phenotypes to objectively stratify pain perception based on the presence of a specific COMT haplotype diploid pairs. Exemplary odds ratios (OR) were determined to quantify the impact of a COMT haplotype diploid pairs to be associated with the presence or absence of a reduced pain perception (PP), neutral PP or elevated PP. The results of the mathematical analysis on the impact of COMT haplotype diploid pairs on pain perception predisposition are listed in Table 6 below.

TABLE 6
Results for COMT Haplotype Diplotype Polymorphisms

| | COMT | | | |
|---|---|---|---|---|
| | Haplotype | General Impact | Exemplary Description | Exem- |
| | Diploid | to Pain | of Pain Perception | plary |
| No. | Polymorphism | Perception (PP) | Predisposition (PPP) | OR |
| 1 | LPS/LPS | Low Pain | Low Perception/High | −2.000 |
| | | Sensitivity | Tolerance | |
| 2 | LPS/APS | Moderately | Moderate-Low | −1.000 |
| | | Low | Perception/ | |
| | | Sensitivty | Mod.-High Tolerance | |
| 3 | APS/APS | Average | Average Perception/ | 0 |
| | | Sensitivty | Average Tolerance | |
| 4 | LPS/HPS | Moderately | Moderate-High | +1.000 |
| | | High | Perception/ | |
| | | Sensitivty | Mod.-Low Tolerance | |
| 5 | APS/HPS | High | High Perception/Low | +2.000 |
| | | Sensitivity | Tolerance | |
| 6 | HPS/HPS | High | High Perception/Low | +2.000 |
| | | Sensitivity | Tolerance | |

The invention further provides systems and methods which are associated, at least in part, with a determination of the presence or absence of one or more of the polymorphisms listed in Tables 1 through 6. For example, information obtained using the diagnostic assays described herein is useful for determining a likely pain perception predisposition in a patient and a likely response if administered a medication and/or a likelihood of a positive response to the treatment. Based on this prognostic information, a clinician can recommend a therapeutic protocol useful for treating an individual based on their genetic predisposition pain perception, or adjust a previously administered therapy to accommodate the patient's sensitivities.

In addition, knowledge of the identity of a particular polymorphism in an individual's genetic profile allows customization of medication or therapy based on the particular individual's genetic profile. For example, an individual's genetic profile can enable a doctor: 1) to more effectively prescribe a drug that will address the patient's medical condition; 2) to better determine an appropriate dosage of a particular drug and 3) to identify novel targets for drug development. The ability to target populations expected to show the highest clinical benefit, based on the genetic profile, can enable: 1) the repositioning of marketed drugs with disappointing market results; 2) the rescue of drug candidates whose clinical development has been discontinued as a result of safety or efficacy limitations, which may be patient subgroup-specific; and 3) an accelerated and less costly development for drug candidates and more optimal drug labeling.

Detection of point mutations or other types of allelic variants can be accomplished by molecular cloning of the specified allele and subsequent sequencing of that allele using techniques known in the art. Alternatively, the gene sequences can be amplified directly from a genomic DNA preparation from the DNA sample using PCR, and the sequence composition is determined from the amplified product. As described more fully below, numerous methods are available for analyzing a subject's DNA for mutations at a given genetic locus such as the gene of interest.

A detection method is allele specific hybridization using probes overlapping the polymorphic region and having, for example, about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described, e.g., in Cronin et al. (1996) Human Mutation 7:244.

In other detection methods, it is necessary to first amplify at least a portion of the gene of interest prior to identifying the allelic variant. Amplification can be performed, e.g., by PCR, according to methods known in the art. In one embodiment, genomic DNA of a cell is exposed to two PCR primers and amplification for a number of cycles sufficient to produce the required amount of amplified DNA. Alternative amplification methods include: self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In other embodiments, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad. Sci. USA 74:560 or Sanger et al. (1977) Proc. Nat. Acad. Sci. 74:5463. It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO94/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster; U.S. Pat. No. 5,605,798 and International Patent Application No. PCT/US96/03651 entitled DNA Diagnostics Based on Mass Spectrometry by H. Koster; Cohen et al. (1996) Adv. Chromat. 36:127-162; and Griffin et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, analyses where only one nucleotide is detected can be carried out.

Yet other sequencing methods are disclosed, e.g., in U.S. Pat. No. 5,580,732 entitled “Method of DNA Sequencing Employing a Mixed DNA-Polymer Chain Probe” and U.S. Pat. No. 5,571,676 entitled “Method For Mismatch-Directed In Vitro DNA Sequencing.” In some cases, the presence of a specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on base pair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility are used to identify the particular allelic variant. For example, single strand conformation polymorphism (SSCP) may be used to detect differences in electrophoretic mobility between mutant and wild type nucleic acids (Orita et al. (1989) Proc Natl. Acad. Sci. USA 86:2766; Cotton (1993) Mutat. Res. 285:125-144 and Hayashi (1992) Genet. Anal. Tech. Appl. 9:73-79). Single-stranded DNA fragments of sample and control nucleic acids are denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In other embodiments, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Exemplary techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

In another embodiment, identification of an allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on the OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e., digoxigenin and fluorescein, each OLA reaction can be detected by using hapten specific antibodies that are labeled with different enzyme reporters, alkaline phosphatase or horseradish peroxidase. This permits the detection of the two alleles using a high throughput format that leads to the production of two different colors.

The invention further provides methods for detecting a polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such polymorphisms.

In one embodiment, the polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide is present in the polymorphic site of the target molecule was complementary to that of the nucleotide derivative used in the reaction. This method has the advantage that it does not require the determination of large amounts of extraneous sequence data.

In another embodiment, a solution-based method is used for determining the identity of the nucleotide of the polymorphic site. Cohen et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al., supra, is preferably a heterogeneous phase assay, in which the primer or the target molecule is immobilized to a solid phase.

Several primer-guided nucleotide incorporation procedures for assaying polymorphic sites in DNA have been described (Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

The genotype information obtained from analyzing a sample of a patient's genetic material may be utilized, according to the principles of the invention, to predict whether a patient has a level of risk associated with taking a medication for treating medical condition. The risk may be associated with a side effect the patient may be susceptible to developing, an efficacy of the drug to the patient specifically or some combination thereof.

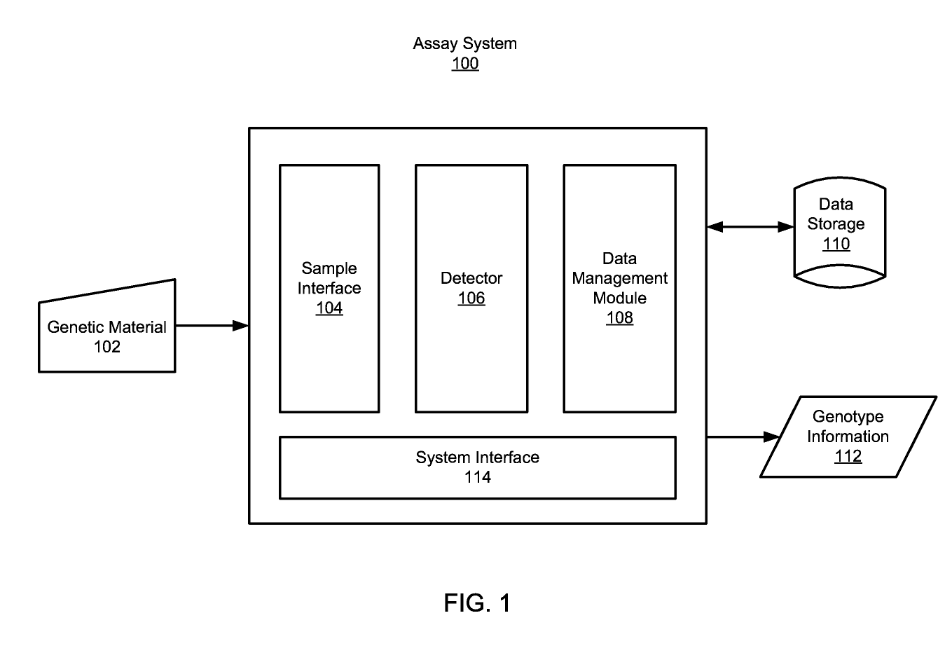

Referring to FIG. 1, depicted is an assay system 100. An assay system, such as assay system 100, may access or receive a genetic material, such as genetic material 102. The sample of genetic material 102 can be obtained from a patient by any suitable manner. The sample may be isolated from a source of a patient's DNA, such as saliva, buccal cells, hair roots, blood, cord blood, amniotic fluid, interstitial fluid, peritoneal fluid, chorionic villus, semen, or other suitable cell or tissue sample. Methods for isolating genomic DNA from various sources are well-known in the art. Also contemplated are non-invasive methods for obtaining and analyzing a sample of genetic material while still in situ within the patient's body.

The genetic material 102 may be received through a sample interface, such as sample interface 104 and detected using a detector, such as detector 106. A polymorphism may be detected in the sample by any suitable manner known in the art. For example, the polymorphism can be detected by techniques, such as allele specific hybridization, allele specific oligonucleotide ligation, primer extension, minisequencing, mass spectroscopy, heteroduplex analysis, single strand conformational polymorphism (SSCP), denaturing gradient gel electrophoresis (DGGE), oligonucleotide microarray analysis, temperature gradient gel electrophoresis (TGGE), and combinations thereof to produce an assay result. The assay result may be processed through a data management module, such as data management module 108, to produce genotype information 112. The genotype information 112 may include an assay result on whether the patients has a genotype including one or more of the allelic variants listed in Table I above. The genotype information 112 may be stored in data storage 110 or transmitted to another system or entity via a system interface 114.

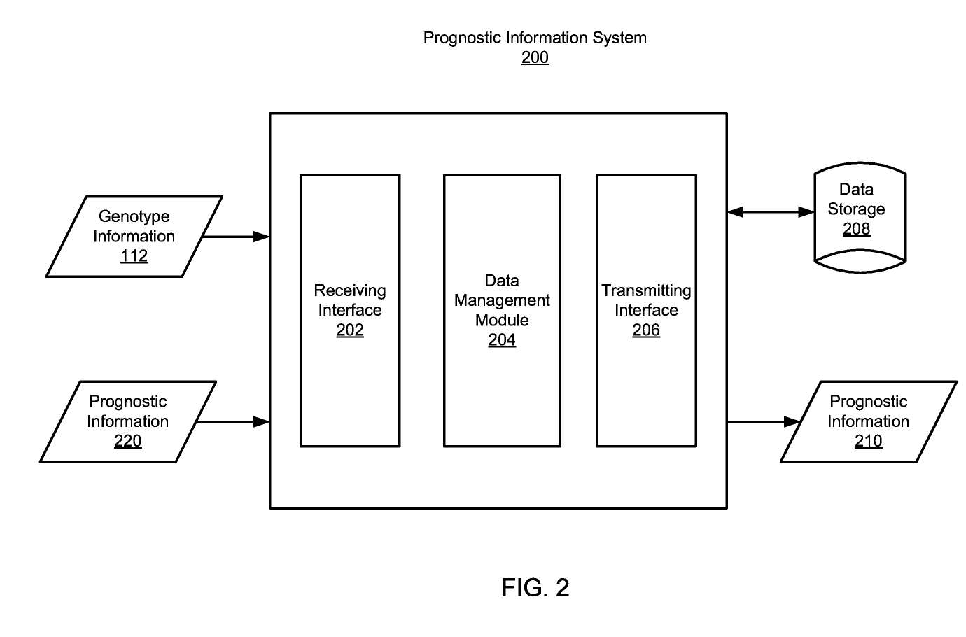

Referring to FIG. 2, depicted is a prognostic information system 200. The prognostic information system 200 may be remotely located away from the assay system 100 or operatively connected with it in an integrated system. The prognostic information system 200 receives the genotype information 112 through a receiving interface 202 for processing at a data management module 204 to generate prognostic information 210. The data management module 204 may utilize one or more algorithms described in greater detail below to generate prognostic information 210. The prognostic information 210 may be stored in data storage 208 or transmitted via a transmitting interface 206 to another system or entity. The transmitting interface 206 may be the same or different as the receiving interface 202. Furthermore, the system 200 may receive prognostic information 220 prepared by another system or entity. Prognostic information may be utilized, in addition to or in the alternative, to genotype information 112 in generating prognostic information 210.

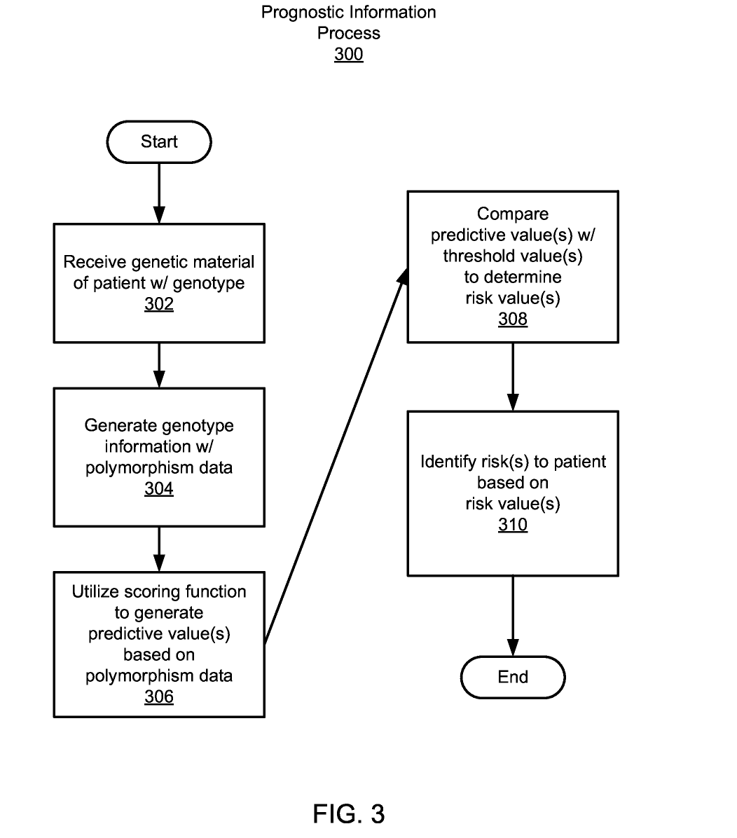

Referring to FIG. 3, depicted is a prognostic information process 300 which may be utilized for preparing information, such as genotype information 112 and prognostic information 210, utilizing an assay system, such as assay system 100 and/or a prognostic information system, such as prognostic information system 200, according to an embodiment. The steps of process 300, and other methods described herein, are described by way of example with the assay system 100 and the prognostic information system 200. The process 300 may be performed with other systems as well.

After process start, at step 302, a sample of genetic material of a patient is obtained as it is received at the sample interface 106. The sample interface can be any type of receptacle for holding or isolating the genetic material 102 for assay testing.

At step 304, the genetic material 102 is tested utilizing the detector 106 in assay system 100 to generate genotype information 112. The detector 106 may employ any of the assay methodologies described above to identify allelic variants in the genetic material 102 and generate the genotype information 112 including polymorphism data associated with one or more of the DNA polymorphisms described above in Tables 1, 3 and 4, and pain perception predisposition activity associated with the DNA polymorphisms described above in Tables 2, 3 and 5. The sex phenotype information described in Table 3 may alternatively be obtained through input of sex information provided by the patient in an input survey. The data management module 108, utilizing a processor in an associated platform such as described below, may store the genotype information 112 on the data storage 110 and/or transmit the genotype information 112 to another entity or system, such as prognostic information system 200 where it is received at receiving interface 202 for analysis.

At step 306, the genotype information 112 can be analyzed utilizing a processor in an associated platform, such as described below, by using an algorithm which may be programmed for processing through data management module 204. The algorithm may utilize a scoring function to generate predictive values based on the polymorphism data in the genotype information 112. Different algorithms may be utilized to assign predictive values and aggregate values.

For example, an additive effect algorithm may be utilized to generate an analysis of a patient's genetic pain perception predisposition. In the additive effect algorithm, polymorphism data in the genotype information obtained from analyzing a patient's genetic material is utilized to indicate the active polymorphisms identified from a patient's genotype information. A tested polymorphism may be determined to be (1) absent or present in either (2) a heterozygous or (3) a homozygous variant in the patient's genotype. According to the additive effect algorithm, the polymorphisms identified from a patient's genotype information are each assigned a real value, such as an Odds Ratio (OR), depending on how the polymorphisms appears in the patient's genotype.

For example, if the DRD1-ANC polymorphism appears in the genotype information generated based on a DNA sample, a value of −0.6445 (the OR associated with DRD1-ANC in Table 1) may be assigned as a predictive value associated with the DRD1-ANC diploid polymorphism. The scoring function associated with the −0.6445 predictive value is the multinomial logistic regression analysis performed using SPSS to generate the OR associated with DRD1-ANC in Table 1. Other scoring functions may also be utilized as long as the predictive value generated reflects a reduced pain perception (PP) associated with the DRD1-ANC diploid polymorphism.

In another example, the DRD1-ANC (OR=−0.6445), the COMT(2)-HET (OR=0), the SLC6A4-NONA (OR=+1.405), the OPRK1-NONA (OR=−0.6560), the DRD2-ANC (OR=+1.293), and the MTHFR-HET (OR=0) polymorphisms appear in the genotype information generated based on a DNA sample. The aggregate value of the diploid polymorphism results is +1.3975 [i.e., −0.6445+0+1.405+(−0.6560)+1.293+0]. According to the example, a threshold value of zero is assigned for determining an overall predisposition to perceiving pain based on the aggregate value being above or below the threshold value. A risk value may correspond with real number associated with the aggregate value, the positive or negative nature of the real number, or both. Thus, the aggregate value of the sum of the odds ratios being positive, the prognostic information for the patient reflects that the individual is predicted to have an elevated predisposition to perceiving pain (i.e., a higher than average pain perception or lower than average pain tolerance) based on their genotype information.

In another example utilizing the same DNA sample the patient is also identified as a male (OR=+1.345) having the LPS COMT haplotype appear in the genotype information generated based on a DNA sample. In this example, the aggregate value of the diploid polymorphism results is +1.3975. However, this aggregate value is further modified with the predictive value for the male sex phenotype (OR=−1.345) and LPS COMT haplotype (OR=−1.000). The aggregate value of the all the polymorphism results according to this example is therefore, −0.9475 [i.e., +1.3975+(−1.345)+(−1.000)]. Thus, the aggregate value of the odds ratios taken from Tables 1, 3 and 5 as predictive values being negative, the prognostic information for the patient is that the individual, based on all the factors considered, is predicted to have an reduced predisposition to perceiving pain (i.e., a lower than average pain perception or higher than average pain tolerance) based on their genotype information and/or including sex phenotype.

### Exemplary Algorithm(s)

According to an exemplary embodiment, an algorithm may be utilized to “score” a subject's perception to pain based on the subject's genetic information and/or non-genetic characteristics or phenotypes. In the algorithm, various criteria may be applied including assigning one or more values based on an identification of a presence or absence of select genetic criteria and/or non-genetic characteristics or phenotypes to provide an algorithm which produces a score associated with a subject's predicted pain perception.

To gather data for the algorithm, one or more of the SNP Diploid Polymorphisms, such as those listed in Table 1, may be tested and/or analyzed to produce one or more values associated with the presence or absence of the SNP Diploid Polymorphisms. In another example, one or more of the characteristic phenotypes in Tables 3A-3I may be tested and/or analyzed to produce one or more values associated with the presence or absence of the characteristic phenotypes in Tables 3A-3I. In another example, one or more of the COMT Haplotype Polymorphisms in Table 5 may be tested and/or analyzed to produce one or more values associated with the presence or absence of the COMT Haplotype Polymorphisms. In another example, a Diploid Pair the COMT Haplotypes may be tested and/or analyzed to produce one or more values associated with the presence or absence of the Diploid Pair of the COMT Haplotype Polymorphisms. Other factors, such as other SNP Diploid Polymorphisms, other characteristic phenotypes such as those based on a demographic may be tested and/or analyzed to produce one or more values associated with the presence or absence of the other SNP Diploid Polymorphisms, other characteristic phenotypes. In addition, a combination of the various SNP Diploid Polymorphisms, the characteristic phenotypes, the COMT Haplotype Polymorphisms, the Diploid Pair the COMT Haplotypes and/or other factors may be tested and/or analyzed to provide values associated with the various factors being tested.

The values produced are based on results of the various tests. The values may be factored into an algorithm to “score” a subject's perception to pain based on the subject's genetic information and/or non-genetic characteristics or phenotypes. The algorithm may compute a composite score based on the results of the individual tests. The composite score may be calculated based on a simple additive analysis of the individual scores which may be compared with a threshold value for determining pain perception based on the additive score. In addition or in the alternative, more complex functions may be utilized to process the values developed from the testing results, such as one or more weighting factor(s) which are applied to one or more of the individual values based on various circumstances, such as if a subject was tested using specific equipment, a temporal condition, etc.

### DETAILED EXAMPLE

A subject's genetic sample is assayed for genetic information based on the following SNPS to identify the specific diploid alleles associated with each SNP:

| | | SNP Cluster |
|---|---|---|
| Gene Name | Abbr. | Report (rs #) |
| Serotonin Transporter (solute carrier | SLC6A4 | rs140701 |
| family 6 (neurotransmitter transporter), | | |
| member 4) | | |
| 5-hydroxytryptamine (serotonin) receptor | HTR2A | rs7997012 |
| 2A, G protein-coupled | | |
| Dopamine Beta-Hydroxylase | DBH | rs1611115 |
| Methylelenetetrahdrofolate reductase | MTFHR | rs1801133 |
| (GABA)-A receptor, gamma 2 | GABRG2 | rs211014 |
| Opioid receptor, mu 1 | OPRM1 | rs1799971 |
| Catechol-O-Methyltransferase | COMT | rs4680 |
| Catechol-O-Methyltransferase | COMT | rs6269 |
| Catechol-O-Methyltransferase | COMT | rs4633 |
| Catechol-O-Methyltransferase | COMT | rs4818 |

The results of the assays on the SNPs in the genes SLC6A4, 5-HTR2A, DBH, MTFHR, GABRG2 and OPRM1 identify the SNP diploid polymorphism in each gene associated with an elevated pain perception, as identified in Table 2 above. In the algorithm, an elevated pain perception SNP diploid polymorphism from Table 2 is assigned a value of 1, while any other SNP diploid polymorphism of the same SNP in Table 2 is assigned a value of 0.

Furthermore, the results of the assays on the four SNPs in the COMT gene identify the COMT haplotype diploid polymorphism is HPS/HPS, which is associated with high sensitivity to pain and a pain perception that is high perception/low tolerance as shown in Table 6. According to the algorithm of the example, the COMT haplotype diploid polymorphisms shown in Table 6 are assigned a value as follows: LPS/LPS=1, APS/LPS=2, APS/APS=3, HPS/LPS=4, HPS/APS=5, and HPS/HPS=5. Since the assay revealed the subject has the HPS/HPS COMT haplotype diploid polymorphism, a value of 5 is assigned for the algorithm.

A review of the subject's medical records reveals the subject is a hispanic female, aged 35 and has a history of depression and anxiety disorder. Accordingly, according to the algorithm, the subject is assigned a value of 1 for being Hispanic, value of 1 for being female, 1 for being within the age group of 18−59, 1 for having a history of depression and 1 for having a history of Anxiety disorder for a total value of 4 for demographic phenotypes. According to another algorithm, one or more indications of a history or diagnosis of one or more of depression, anxiety disorder and/or any of the other mental health phenotypes may be grouped to form a score of 1.

According to the algorithm, the values for individual SNP diploid polymorphisms (SLC6A4=1, 5-HTR2A=1, DBH=1, MTFHR=1, GABRG2=1 and OPRM1=1) have a cumulative value of 6; the HPS/HPS COMT haplotype diploid polymorphism, have a value of 5 and the demographic phenotypes have a cumulative value of 5. Three separate values may considered separately, but according to the algorithm, they may be combined to provide a total score of 16 (6+5+5). In the algorithm, a threshold value of, for example, 8 may be established as a threshold for pain perception analysis for one category, such as the COMT haplotype diploid only, or in another example, for all three categories. As the subject has total score of 16, they have a high pain perception according to the algorithm of the examples. In other embodiments, other values and thresholds may be established, depending upon the desired objectives, such as statistical significance based upon odd ratios utilized to develop a cumulative score for determining pain perception.

In all of the preceding examples, the predictive values and aggregate values generated are forms of prognostic information 210.

At step 310, the result of the comparison obtained in step 308 generates a second form of prognostic information 220. For example, (a) if the determined sum is higher than the threshold value, it can be predicted that the patient is at a higher risk for a negative risk factor associated with prescribing the patient the medication; (b) if the determined sum is at or near the threshold value, it can be predicted that the patient is at a moderate risk for negative risk factors associated with prescribing the patient the medication; and (c) if the determined sum is below the threshold value, it can be predicted that the patient is at a low risk for negative risk factors associated with prescribing the patient the medication.

Also at step 310, the data management module 205 in the prognostic information system 200 identifies a risk to a patient by executing an algorithm, such as the additive effect algorithm described above, and communicating the generated prognostic information 210. The data management module 204, utilizing a processor in an associated platform such as described below, may store the prognostic information 210 on the data storage 208 and/or transmit the prognostic information 210 to another entity or system prior to end of the prognostic information process 300. Other algorithms may also be used in a similar manner to generate useful forms of prognostic information for determining treatment options for a patient.

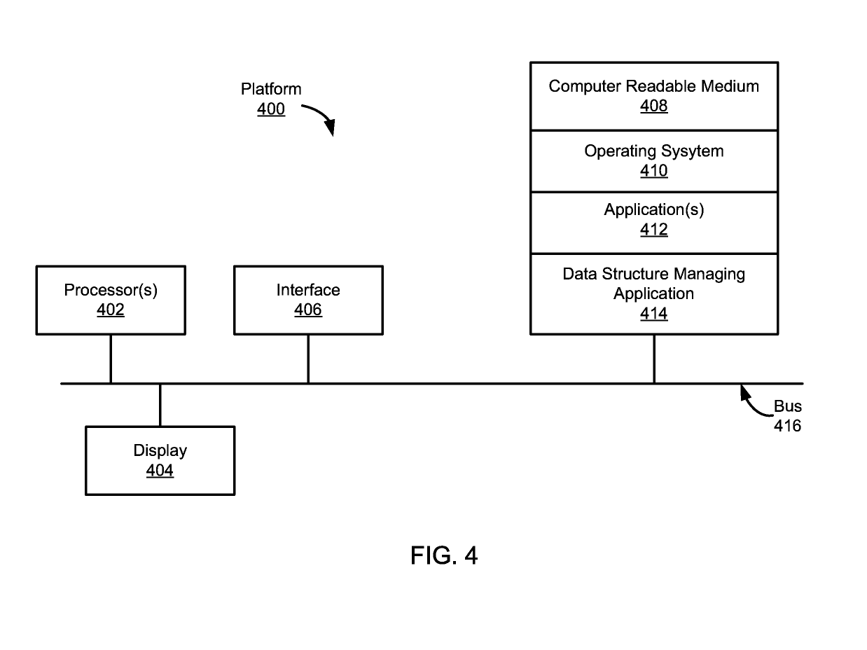

Referring to FIG. 4, there is shown a platform 400, which may be utilized as a computing device in a prognostic information system, such as prognostic information system 200, or an assay system, such as assay system 100. It is understood that the depiction of the platform 400 is a generalized illustration and that the platform 400 may include additional components and that some of the components described may be removed and/or modified without departing from a scope of the platform 400.

The platform 400 includes processor(s) 402, such as a central processing unit; a display 404, such as a monitor; an interface 406, such as a simple input interface and/or a network interface to a Local Area Network (LAN), a wireless 802.11x LAN, a 3G or 4G mobile WAN or a WiMax WAN; and a computer-readable medium (CRM) 408. Each of these components may be operatively coupled to a bus 416. For example, the bus 416 may be an EISA, a PCI, a USB, a FireWire, a NuBus, or a PDS.

A CRM, such as CRM 408 may be any suitable medium which participates in providing instructions to the processor(s) 402 for execution. For example, the CRM 408 may be non-volatile media, such as an optical or a magnetic disk; volatile media, such as memory; and transmission media, such as coaxial cables, copper wire, and fiber optics. Transmission media can also take the form of acoustic, light, or radio frequency waves. The CRM 408 may also store other instructions or instruction sets, including word processors, browsers, email, instant messaging, media players, and telephony code.

The CRM 408 may also store an operating system 410, such as MAC OS, MS WINDOWS, UNIX, or LINUX; application(s) 412, such as network applications, word processors, spreadsheet applications, browsers, email, instant messaging, media players such as games or mobile applications (e.g., “apps”); and a data structure managing application 414. The operating system 410 may be multi-user, multiprocessing, multitasking, multithreading, real-time and the like. The operating system 410 may also perform basic tasks such as recognizing input from the interface 406, including from input devices, such as a keyboard or a keypad; sending output to the display 404 and keeping track of files and directories on CRM 408; controlling peripheral devices, such as disk drives, printers, image capture devices; and for managing traffic on the bus 416. The applications 412 may include various components for establishing and maintaining network connections, such as code or instructions for implementing communication protocols including those such as TCP/IP, HTTP, Ethernet, USB, and FireWire.

A data structure managing application, such as data structure managing application 414 provides various code components for building/updating a computer-readable system architecture, such as for a non-volatile memory, as described above. In certain examples, some or all of the processes performed by the data structure managing application 412 may be integrated into the operating system 410. In certain examples, the processes may be at least partially implemented in digital electronic circuitry, in computer hardware, firmware, code, instruction sets, or any combination thereof.

Although described specifically throughout the entirety of the disclosure, the representative examples have utility over a wide range of applications, and the above discussion is not intended and should not be construed to be limiting. The terms, descriptions and figures used herein are set forth by way of illustration only and are not meant as limitations. Those skilled in the art recognize that many variations are possible within the spirit and scope of the principles of the invention. While the examples have been described with reference to the figures, those skilled in the art are able to make various modifications to the described examples without departing from the scope of the following claims, and their equivalents.

What is claimed is:

1. A method for preparing prognostic information about pain perception, comprising:
providing information, including DNA information, associated with a human subject;
determining from the information that the human subject is characterized by at least two demographic phenotypes, the subject has a subject genotype that includes a COMT haplotype diploid, the subject genotype includes at least two SNP diploids or a combination thereof, by detecting, utilizing a detection technology and the information, a presence or absence from the information of the at least two demographic phenotypes, the COMT haplotype diploid and/or the at least two SNP diploids in the subject genotype,
wherein the COMT haplotype diploid is a combination of two COMT haplotypes selected from an LPS haplotype, an APS haplotype, a HPS haplotype or a combination thereof in the COMT gene,
wherein each of the at least two SNP diploids is a combination of two SNP alleles associated with one SNP location, the SNP alleles selected from
DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, and MTHFR-ANC, MTHFR-HET, and MTHFR-NONA in the MTHFR gene, SLC6A4-ANC, SLC6A4-HET, and SLC6A4-NONA in the SLC6A4 gene, HTR2A-ANC, HTR2A-HET, and HTR2A-NONA in the HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene SLC6A3-ANC, SLC6A3-HET, and SLC6A3-NONA in the SLC6A3 gene,
wherein the at least two demographic phenotypes are selected from race, age, gender, depression and/or other mental health demographic phenotypes.

2. A method of claim 1, further comprising
determining an expected pain perception of the subject based, at least in part, on the detected presence or absence of the at least two demographic phenotypes, the COMT haplotype diploid, the at least two SNP diploids, or the combination thereof based on the information.

3. A method of claim 1, wherein the determined expected pain perception is based, at least in part, on the presence or absence of the COMT haplotype diploid and the at least two demographic phenotypes.

4. A method of claim 1, wherein the determined expected pain perception is based, at least in part, on the presence or absence of the COMT haplotype diploid, the at least two demographic phenotypes and the at least two SNP diploids.

5. A method of claim 1, wherein the determined expected pain perception is based, at least in part, on the presence or absence of at least three demographic phenotypes.

6. A method of claim 1, wherein the determined expected pain perception is based, at least in part, on the presence or absence of at least four demographic phenotypes.

7. A method of claim 1, wherein the determined expected pain perception is based, at least in part, on the presence or absence of at least three SNP diploids.

8. A method of claim 1, wherein the determined expected pain perception is based, at least in part, on the presence or absence of at least four SNP diploids.

9. A method of claim 1, further comprising:
utilizing an algorithm to determine the expected pain perception, the algorithm including assigning one or more respective values to a finding of the presence or absence of the respective demographic phenotypes, the respective COMT haplotype diploid, and/or the respective at least two SNP diploids.

10. A method of claim 9,
wherein the algorithm includes adding the respective values to determine a score and comparing the score to a threshold.

11. A method for utilizing prognostic information about pain perception, comprising:
receiving information, including DNA information, associated with a human subject,
wherein the received information indicates that the human subject is characterized by at least two demographic phenotypes, the subject has a subject genotype that includes a COMT haplotype diploid, the subject genotype includes at least two SNP diploids, or a combination thereof,
wherein the COMT haplotype diploid is a combination of two COMT haplotypes selected from an LPS haplotype, an APS haplotype, a HPS haplotype or a combination thereof in the COMT gene,
wherein each of the at least two SNP diploids is a combination of two SNP alleles associated with one SNP location, the SNP alleles selected from DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, HTR2A-ANC, HTR2A-HET, and HTR2A-NONA in the HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene,
wherein the at least two demographic phenotypes are selected from race, age, gender, depression and/or other mental health demographic phenotypes;
processing the received information utilizing a processor; and
determining a therapy for the human subject based, at least in part, on the processed information.

12. A method of claim 11, further comprising
determining an expected pain perception of the subject based, at least in part, on the presence or absence of the at least two demographic phenotypes, the COMT haplotype diploid, the at least two SNP diploids, or the combination thereof based on the information.

13. A method of claim 12, further comprising
utilizing an algorithm to determine the expected pain perception, the algorithm including assigning one or more respective values to a finding of the presence or absence of the respective demographic phenotypes, the respective COMT haplotype diploid, and/or the respective at least two SNP diploids,
wherein the algorithm includes adding the respective values to determine a score and comparing the score to a threshold.

14. A method of claim 11, wherein the determined expected pain perception is based, at least in part, on the presence or absence of the COMT haplotype diploid and the at least two demographic phenotypes.

15. A method of claim 11, wherein the determined expected pain perception is based, at least in part, on the presence or absence of the COMT haplotype diploid, the at least two demographic phenotypes and the at least two SNP diploids.

16. A method of claim 11, wherein the determined expected pain perception is based, at least in part, on the presence or absence of at least three demographic phenotypes.

17. A method of claim 11, wherein the determined expected pain perception is based, at least in part, on the presence or absence of at least three SNP diploids.

18. A method for performing an assay, comprising:
providing a sample of genetic material of a human subject; and
determining DNA information from the sample, the DNA information including whether a subject genotype of the subject includes a COMT haplotype diploid, the subject genotype of the subject includes at least two SNP diploids, or a combination thereof, by detecting, utilizing a detection technology and the sample, a presence or absence of the COMT haplotype diploid and/or the at least two SNP diploids in the subject genotype,
wherein the COMT haplotype diploid is a combination of two COMT haplotypes selected from LPS haplotype, APS haplotype, HPS haplotype or a combination thereof in the COMT gene,
wherein each of the at least two SNP diploids is a combination of two SNP alleles associated with one SNP location, the SNP alleles selected from
DRD1-ANC, DRD1-HET, and DRD1-NONA in the DRD1 gene, COMT(2)-ANC, COMT(2)-HET, and COMT(2)-NONA in the COMT gene, SLC6A4*-ANC, SLC6A4*-HET, and SLC6A4*-NONA in the SLC6A4 gene, OPRK1-ANC, OPRK1-HET, and OPRK1-NONA in the OPRK1 gene, DRD2-ANC, DRD2-HET, and DRD2-NONA in the DRD2 gene, MTHFR-ANC, MTHFR-HET, and MTHFR-NONA in the MTHFR gene, SLC6A4-ANC, SLC6A4-HET, and SLC6A4-NONA in the SLC6A4 gene, 5-HTR2A-ANC, 5-HTR2A-HET, and 5-HTR2A-NONA in the 5-HTR2A gene, DBH-ANC, DBH-HET, and DBH-NONA in the DBH gene, GABRG2-ANC, GABRG2-HET, and GABRG2-NONA in the GABRG2 gene, OPRM1-ANC, OPRM1-HET, and OPRM1-NONA in the OPRM1 gene, SLC6A3-ANC, SLC6A3-HET, and SLC6A3-NONA in the SLC6A3 gene.

19. A method of claim 18, wherein the determined DNA information is based, at least in part, on the presence or absence of the COMT haplotype diploid and the at least two SNP diploids in the subject genotype.

20. A method of claim 18, wherein the determined DNA information is based, at least in part, on the presence or absence of at least three SNP diploids in the subject genotype.
